(19) United States
(12) Patent Application Publication
(10) Pub. No.: US 2025/0241925 A1
(43) Pub. Date: Jul. 31, 2025

(54) METHODS OF P21 SUPPRESSION FOR BONE REGENERATION AND HEALING

(71) Applicant: UWM Research Foundation, Inc., Milwaukee, WI (US)

(72) Inventors: Priyatha Premnath, Milwaukee, WI (US); Sina Jafari, Greenfield, WI (US)

(21) Appl. No.: 18/855,866

(22) PCT Filed: Apr. 12, 2023
(86) PCT No.: PCT/US2023/018266

§ 371 Date: Oct. 10, 2024

Related U.S. Application Data
(60) Provisional application No. 63/330,431, filed on Apr. 13, 2022.

(51) Int. Cl. A61K 31/5377; A61K 31/341; A61K 31/4439; A61K 31/4704; A61K 31/472; A61K 31/515; A61K 45/06; A61P 19/10

(52) U.S. Cl. CPC A61K 31/5377; A61K 31/341; A61K 31/4439; A61K 31/4704; A61K 31/472; A61K 31/515; A61K 45/06; A61P 19/10

(57) ABSTRACT

Described herein are compositions and methods for accelerating the healing of a bone fracture in a subject, regenerating bone in a subject, and reducing the likelihood of bone loss in an osteoporotic environment of a subject. In some embodiments, the compositions and methods may comprise one or more inhibitors of p21 to suppress p21 gene and protein expression in a subject. In some embodiments, suppression of p21 expression in mesenchymal stem cells (MSCs) in a subject may reduce adipogenic differentiation, increase osteogenic capacity, and increase chondrogenic capacity in the subject.

### CROSS-REFERENCE TO RELATED APPLICATIONS

This application claims priority to U.S. Provisional Pat. App. No. 63/330,431, filed on Apr. 13, 2022, which is incorporated by reference herein in its entirety.

### TECHNICAL FIELD

Described herein are compositions and methods for accelerating the healing of a bone fracture in a subject, regenerating bone in a subject, and reducing the likelihood of bone loss in an osteoporotic environment of a subject. In some embodiments, the compositions and methods may comprise one or more inhibitors of p21 to suppress p21 gene and protein expression in a subject. In some embodiments, suppression of p21 expression in mesenchymal stem cells (MSCs) in a subject may reduce adipogenic differentiation, increase osteogenic capacity, and increase chondrogenic capacity in the subject.

### BACKGROUND

Bones heal completely after fracture, but in cases like advanced age this process is substantially altered resulting in hampered and delayed healing or non-union of bone. This can result in changes in biomechanics of the bone leading to other complications and ultimately affect quality of life. It has been demonstrated that mesenchymal stem cells (MSCs) play a vital role in the bone healing process, and in advanced age, MSCs have reduced bone forming capacity. Current treatment strategies focus on downstream osteogenic factors, often showing limited success.

What is needed are methods and compositions to improve bone healing after fracture, or implants, improve bone regeneration, and prevent bone loss in osteoporotic environments or during chemotherapy.

### SUMMARY

One embodiment described herein is a method for accelerating the healing of a bone fracture, implant, or joint replacement in a subject, the method comprising: administering to the subject a therapeutically effective amount of one or more inhibitors of p21 using a dosing regimen comprising 5 mg/kg to 100 mg/kg for a period of time of 1 day to 6 months, thereby accelerating the healing of the bone fracture in the subject. In one aspect, the one or more inhibitors of p21 comprise UC2288, Butyrolactone I, Sorafenib, LLW10, Daprodustat, Vadadustat, Molidustat, Roxadustat, Desidustat, or combinations thereof. In another aspect, the one or more inhibitors of p21 comprise UC2288 having the structure:

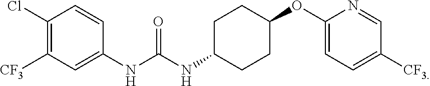 [SMILES: O=C(Nc1ccc(Cl)c(C(F)(F)F)c1)N[C@H]1CC[C@H](Oc2ccc(C(F)(F)F)cn2)CC1]

In another aspect, the one or more inhibitors of p21 suppress p21 gene expression and protein expression in the subject. In another aspect, the one or more inhibitors of p21 suppress p21 gene expression and protein expression in mesenchymal stem cells (MSCs) in the subject. In another aspect, suppression of p21 gene expression and protein expression in MSCs in the subject reduces adipogenic differentiation, increases osteogenic capacity, and increases chondrogenic capacity in the subject. In another aspect, the subject is ≥50 years of age. In another aspect, the subject is ≥75 years of age. In another aspect, the subject is diabetic or has an insulin resistance disorder In another aspect, the one or more inhibitors of p21 are administered to the subject at a situs of the bone fracture, implant, or joint replacement. In another aspect, the one or more inhibitors of p21 are systemically administered to the subject. In another aspect, the dosing regimen comprises a single dose or a plurality of doses of the therapeutically effective amount of the one or more inhibitors of p21.

Another embodiment described herein is a method for regenerating bone or improving bone strength in a subject, the method comprising: administering to the subject a therapeutically effective amount of one or more inhibitors of p21 using a dosing regimen comprising 5 mg/kg to 100 mg/kg for a period of time of 1 day to 6 months, thereby regenerating bone in the subject. In one aspect, the one or more inhibitors of p21 comprise UC2288, Butyrolactone I, Sorafenib, LLW10, Daprodustat, Vadadustat, Molidustat, Roxadustat, Desidustat, or combinations thereof. In another aspect, the one or more inhibitors of p21 comprise UC2288 having the structure:

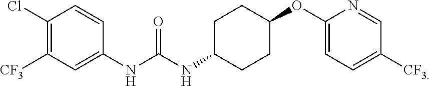 [SMILES: O=C(Nc1ccc(Cl)c(C(F)(F)F)c1)N[C@H]1CC[C@H](Oc2ccc(C(F)(F)F)cn2)CC1]

In another aspect, the one or more inhibitors of p21 suppress p21 gene expression and protein expression in the subject. In another aspect, the one or more inhibitors of p21 suppress p21 gene expression and protein expression in mesenchymal stem cells (MSCs) in the subject. In another aspect, suppression of p21 gene expression and protein expression in MSCs in the subject reduces adipogenic differentiation, increases osteogenic capacity, and increases chondrogenic capacity in the subject. In another aspect, the subject is ≥50 years of age. In another aspect, the subject is ≥75 years of age. In another aspect, the subject is diabetic or has an insulin resistance disorder. In another aspect, the one or more inhibitors of p21 are administered to the subject at a situs for bone regeneration or improving bone strength. In another aspect, the one or more inhibitors of p21 are systemically administered to the subject. In another aspect, the dosing regimen comprises a single dose or a plurality of doses of the therapeutically effective amount of the one or more inhibitors of p21.

Another embodiment described herein is a method for reducing the likelihood of bone loss in an osteoporotic environment or during chemotherapeutic treatment of a subject, the method comprising: administering to the subject a therapeutically effective amount of one or more inhibitors of p21 using a dosing regimen comprising 5 mg/kg to 100 mg/kg for a period of time of 1 day to 6 months, thereby reducing the likelihood of bone loss in the subject. In one aspect, the one or more inhibitors of p21 comprise UC2288, Butyrolactone I, Sorafenib, LLW10, Daprodustat, Vadadustat, Molidustat, Roxadustat, Desidustat, or combinations thereof. In another aspect, the one or more inhibitors of p21 comprise UC2288 having the structure:

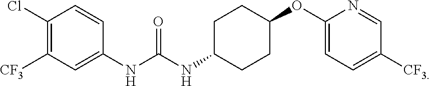 [SMILES: O=C(Nc1ccc(Cl)c(C(F)(F)F)c1)N[C@H]1CC[C@H](Oc2ccc(C(F)(F)F)cn2)CC1]

In another aspect, the one or more inhibitors of p21 suppress p21 gene expression and protein expression in the subject. In another aspect, the one or more inhibitors of p21 suppress p21 gene expression and protein expression in mesenchymal stem cells (MSCs) in the subject. In another aspect, suppression of p21 gene expression and protein expression in MSCs in the subject reduces adipogenic differentiation, increases osteogenic capacity, and increases chondrogenic capacity in the subject. In another aspect, the subject is ≥50 years of age. In another aspect, the subject is ≥75 years of age. In another aspect, the subject is diabetic or has an insulin resistance disorder. In another aspect, the one or more inhibitors of p21 are administered to the subject at a situs of the osteoporotic environment. In another aspect, the one or more inhibitors of p21 are systemically administered to the subject. In another aspect, the dosing regimen comprises a single dose or a plurality of doses of the therapeutically effective amount of the one or more inhibitors of p21. In another aspect, the one or more inhibitors of p21 are systemically administered to the subject with a chemotherapeutic.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show MTT assay results of MSCs treated with UC2288 concentrations of 2.5 μM, 5 μM, and 10 μM for 24 hours (FIG. 1A) and 48 hours (FIG. 1B).

FIG. 2A-B show the effects of UC2288 treatment by staining for calcium deposits by osteoblasts differentiated from MSCs. FIG. 2A shows MSCs treated with 10 M UC2288. FIG. 2B shows MSCs treated with the positive control of DMEM media. Greater staining was observed for MSCs treated with 10 μM UC2288. The scale bar represents 2 mm.

FIG. 3 shows a graph of the osteogenic staining capacity of MSCs after interaction with UC2288 at various concentrations.

FIG. 4 shows a schematic of the experimental workflow for injections of UC2288 into the callus of mice with fractured tibiae.

FIG. 5 shows an image of the soft tissue and bone volume differences in fractured tibiae of mice treated with control or UC2288. Mice injected with five 10 μM doses of UC2288 showed more bone formation/healing.

FIG. 6A-C show results gene expression studies. FIG. 6A shows results for Sp7 and demonstrates increased osteogenic gene expression at all concentrations as compared to the control. When compared to the osteogenic positive control, both 5 and 10 μM had increased osteogenic potential. FIG. 6B shows results for RunX2 and demonstrates increased osteogenic potential compared to controls. However, expression was less than the osteogenic control. The 5 μM sample had most significant effect on osteogenic markers compared to 2.5 and 10 μM. FIG. 6C shows expression of p21 in HuMSCs. While 2.5 and 5 μM showed reduced p21 expression, 10 μM showed increased expression.

FIG. 7A-D show microCT scan images of mouse tibia treated with five 10 μM doses of UC2288 after fracture (FIG. 7A, C) or controls (FIG. 7B, D). The blue contour in the sample (FIG. 7A) and control (FIG. 7B) images shows callus formation in the area of fracture and the green contour shows bone being regenerated. FIG. 7C-D show black and white images show where bone was automatically thresholded for the treated sample (FIG. 7C) or the control (FIG. 7D).

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing, reducing the progression of, or reducing the likelihood of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. In another embodiment, the subject is a mouse.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, reducing the likelihood, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “implant” refers to a bone implant or bone graft. The bone implant or graft replaces missing bone with material from a subject's own body or from an artificial, synthetic, or other natural substitute. Bone implanting or grafting is possible because bone tissue has the ability to regenerate completely if provided the space into which it has to grow. In some embodiments of the present invention, compositions and methods are described for accelerating the healing of an implant in a subject.

As used herein, “joint replacement” refers to a replacement of a joint produced by removing part or all of an arthritic or damaged joint and installing hardware to allow the limb to move without pain or limitations. Parts of the arthritic or damaged joint are removed and replaced with hardware comprising a metal, plastic, ceramic, or combination of these materials, where the hardware is called a prosthesis. The prosthesis is designed to replicate the movement of a normal, healthy joint. In some embodiments of the present invention, compositions and methods are described for accelerating the healing of a joint replacement in a subject.

As used herein, “improving bone strength” or “improved bone strength” refers to a measured or observed increase in a bone's resistance to fracture. Bone strength is related to, but not equivalent with, bone mineral density. Bone mineral density can be a strong predictor of fracture, but there are also other factors, such as bone structure, bone remodeling, and bone quality to consider. Bone strength may also be determined by bone geometry, cortical thickness and porosity, trabecular bone morphology, and intrinsic properties of bony tissue. In some embodiments of the present invention, compositions and methods are described for improving bone strength in a subject.

As used herein, “accelerating the healing of a bone fracture, implant, or joint replacement” or “accelerated healing of a bone fracture, implant, or joint replacement” refers to a reduction in the total amount of time required for a bone fracture, implant, or joint replacement to fully heal in a subject as assessed by the subject's level of bone strength, bone mass, pain, or mobility, or by other means well known in the art. In some embodiments of the present invention, compositions and methods are described for accelerating the healing of a bone fracture, implant, or joint replacement in a subject.

As used herein, “regenerating bone” or “bone regeneration” refers to a proliferative physiological process of new bone cell formation (e.g., osteocytes) in response to injury, as well as during skeletal development or continuous remodeling throughout adult life. Typically, the most common form of bone regeneration is in fracture healing, during which the pathway of normal fetal skeletogenesis, including intramembranous and endochondral ossification, is recapitulated. The bone regeneration and healing process may be determined by the periosteum (the connective tissue membrane covering the bone), as the periosteum is one source of precursor cells that develop into chondroblasts and osteoblasts that are important in the healing of bone. Other potential sources of precursor cells are the bone marrow, endosteum, small blood vessels, and fibroblasts. In some embodiments of the present invention, compositions and methods are described for regenerating bone in a subject.

As used herein, “reducing the likelihood of bone loss in an osteoporotic environment” or “reduced likelihood of bone loss in an osteoporotic environment” refers to a reduced probability of a subject experiencing bone loss or bone fracture in an osteoporotic environment. The osteoporotic environment may comprise a specific bone or tissue site in a subject's body where osteoporosis is occurring. For example, the osteoporotic environment may comprise a site of decreased bone mineral density and bone mass, or a site of decreased bone strength or modified bone structure. In some embodiments of the present invention, compositions and methods are described for reducing the likelihood of bone loss in an osteoporotic environment in a subject.

As used herein, “osteogenic capacity” refers to the ability of mesenchymal stem cells (MSCs) to differentiate into osteoblasts. Osteoblasts are cells that lay down new bone. Osteoblasts then differentiate into osteocytes. Osteocytes are bone cells that reside within the bone matrix. They typically have an oblate shape with dendritic processes and are the most commonly found bone cell in a mature bone structure. In some embodiments of the present invention, compositions and methods are described for increasing osteogenic capacity.

As used herein, “chondrogenic capacity” refers to the ability of MSCs to differentiate into chondrocytes. Chondrocytes are cells that produce and maintain cartilage tissue and the cartilaginous matrix. In some embodiments of the present invention, compositions and methods are described for increasing chondrogenic capacity.

p21-null mice (−/−) show accelerated bone healing after injury. See Premnath et al., BMC Musculoskeletal Disord. 18: 435 (2017). Mesenchymal stem cells at the site of injury were reported to be a contributing factor potentially via improved chondrogenesis. For bone healing in aged mice, p21 expression is increased compared to young mice.

Described herein are compositions and methods for accelerating or promoting bone healing using a small-molecule, drug-based approach to activate cell cycle. UC2288 is a cell-permeable p21 inhibitor. Inhibition of p21 may improve and accelerate bone healing outcomes after injury. For example, the use of p21 suppressors such as UC2288 may improve bone healing after fracture.

Embodiments described herein relate to a method of suppressing p21 gene and protein expression to aid in bone healing and regeneration. In one aspect, p21 suppression can be accomplished through the use of inhibitors of p21, including but not limited to those shown in Table 1.

TABLE 1
p21 Inhibitors
UC2288
1-(4-chloro-3-(trifluoromethyl)phenyl)-3-((1r,4r)-4-((5-(trifluoromethyl)pyridin-2-
yl)oxy)cyclohexyl)urea

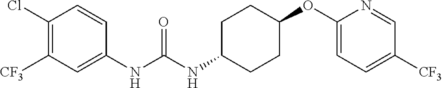 [SMILES: O=C(Nc1ccc(Cl)c(C(F)(F)F)c1)N[C@H]1CC[C@H](Oc2ccc(C(F)(F)F)cn2)CC1]

Butyrolactone I
(2R)-2,5-dihydro-4-hydroxy-2-[[4-hydroxy-3-(3-methyl-2-buten-1-yl)phenyl]methyl]-3-(4-
hydroxyphenyl)-5-oxo-2-furancarboxylic acid, methyl ester

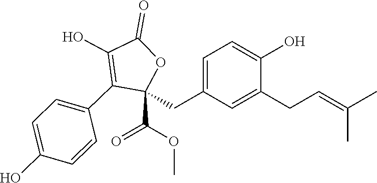 [SMILES: COC(=O)[C@]1(Cc2ccc(O)c(CC=C(C)C)c2)OC(=O)C(O)=C1c1ccc(O)cc1]

Sorafenib (Nexavar)
4-[4-[[4-chloro-3-(trifluoromethyl)phenyl]carbamoylamino]phenoxy]-N-methyl-pyridine-2-
carboxamide

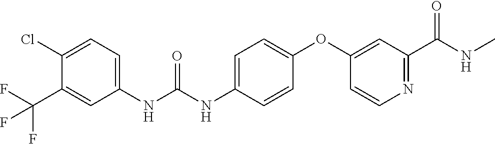 [SMILES: CNC(=O)c1cc(Oc2ccc(NC(=O)Nc3ccc(Cl)c(C(F)(F)F)c3)cc2)ccn1]

LLW10
3-(2-(anthracen-9-yl)-1-(3-(2-methylpiperidin-1-yl)propyl)-1H-benzo[d]imidazol-5-yl)-3-(3-(o-
tolyl)ureido)propanamide. See Park et al., Cancer Biol. Ther. 7(12): 2015-2022 (2008).

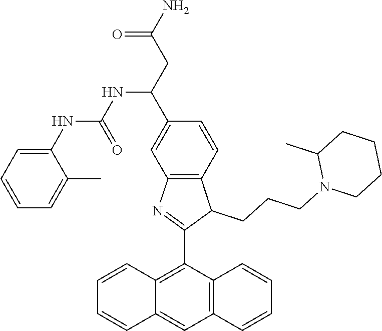 [SMILES: Cc1ccccc1NC(=O)NC(CC(N)=O)c1ccc2c(c1)N=C(c1c3ccccc3cc3ccccc13)C2CCCN1CCCCC1C]

Daprodustat (Jesduvroq, Duvroq)
2-[(1,3-dicyclohexyl-2,4,6-trioxo-1,3-diazinane-5-carbonyl)amino]acetic acid

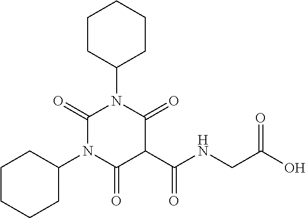 [SMILES: O=C(O)CNC(=O)C1C(=O)N(C2CCCCC2)C(=O)N(C2CCCCC2)C1=O]

Vadadustat (PG-1016548; AKB-6548)
2-([5-(3-Chlorophenyl)-3-hydroxypyridine-2-carbonyl]amino)acetic acid

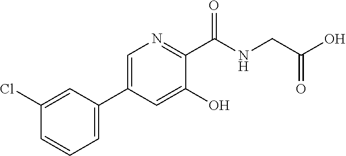 [SMILES: O=C(O)CNC(=O)c1ncc(-c2cccc(Cl)c2)cc1O]

Molidustat (Bay 85-3934)
2-(6-Morpholin-4-ylpyrimidin-4-yl)-4-(triazol-1-yl)-1H-pyrazol-3-one

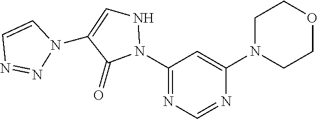 [SMILES: O=c1c(-n2ccnn2)c[nH]n1-c1cc(N2CCOCC2)ncn1]

Roxadustat (Evrenzo; FG-4592, ASP1517, AZD9941)
2-[(4-Hydroxy-1-methyl-7-phenoxyisoquinoline-3-carbonyl)amino]acetic acid

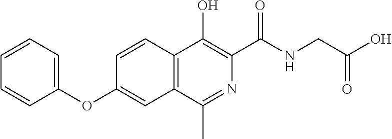 [SMILES: Cc1nc(C(=O)NCC(=O)O)c(O)c2ccc(Oc3ccccc3)cc12]

Desidustat (ZYAN1; Oxemia)
2-[[1-(Cyclopropylmethoxy)-4-hydroxy-2-oxoquinoline-3-carbonyl]amino]acetic acid

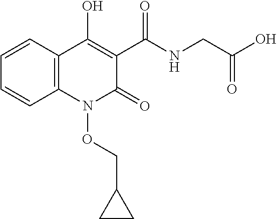 [SMILES: O=C(O)CNC(=O)c1c(O)c2ccccc2n(OCC2CC2)c1=O]

In one aspect, the p21-inhibitor is 1-(4-chloro-3-(trifluoromethyl)phenyl)-3-((1r,4r)-4-((5-
(trifluoromethyl)pyridin-2-yl)oxy)cyclohexyl)urea:

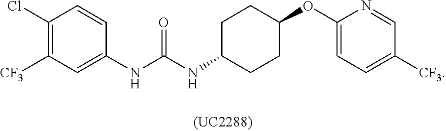 [SMILES: O=C(Nc1ccc(Cl)c(C(F)(F)F)c1)N[C@H]1CC[C@H](Oc2ccc(C(F)(F)F)cn2)CC1]

In another aspect, UC2288 is administered to accelerate bone healing and regeneration. UC2288 may be administered systematically, at the site of injury, or be impregnated into a biodegradable extended-release depot or gauze that is applied to a bone injury or surgical site.

Another embodiment described herein is a method of suppressing p21 gene and protein expression to aid in bone healing and regeneration in geriatric mammals.

In one aspect, this disclosure describes a method of treatment using a small molecule to suppress p21 gene and protein expression to aid in bone healing and regeneration.

In another aspect, this disclosure describes a method of treatment using the molecule UC2288 to suppress p21 gene and protein expression to aid in bone healing and regeneration.

In another aspect, this disclosure describes a method of suppressing p21 in mesenchymal stem cells (MSCs) where p21 is overexpressed due to aging.

In another aspect, this disclosure describes a method of suppressing p21 in MSCs to reduce adipogenic differentiation.

In another aspect, this disclosure describes a method of suppressing p21 in MSCs to improve osteogenic capacity.

In another aspect, this disclosure describes a method of suppressing p21 in MSCs to improve chondrogenic capacity.

In another aspect, this disclosure describes a method of treatment using UC2288 or a p21 suppression agent to prevent loss of bone in an osteoporotic environment.

In another aspect, this disclosure describes a method of treatment using UC2288 or a p21 suppression agent to improve bone formation during endochondral ossification and/or intramembranous ossification.

In some embodiments of the present invention, a subject is administered a therapeutically effective amount of one or more inhibitors of p21 using a specific dosing regimen. In one aspect, the dosing regimen comprises a single dose of the therapeutically effective amount of the one or more inhibitors of p21 administered at a single point in time. In another aspect, the dosing regimen comprises a plurality of doses of the therapeutically effective amount of the one or more inhibitors of p21 administered over a period of time. For example, in various nonlimiting embodiments, one or more inhibitors of p21 as described herein may be administered to a subject once a day (SID/QD), twice a day (BID), three times a day (TID), four times a day (QID), or more, so as to administer a therapeutically effective amount of the one or more inhibitors of p21 to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, one or more inhibitors of p21 are administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof.

The actual dosing regimen can depend upon many factors, including but not limited to the judgment of a trained physician, the overall condition of the subject, the age of the subject, and the specific type of bone condition. The actual dosage can also depend on the determined experimental effectiveness of the specific inhibitors of p21 that are administered. For example, the dosage may be determined based on in vitro responsiveness of relevant cultured cells or artificial bone/tissue models, or in vivo responses observed in appropriate animal models or human studies.

One embodiment described herein is a method for accelerating the healing of a bone fracture, implant, or joint replacement in a subject, the method comprising: administering to the subject a therapeutically effective amount of one or more inhibitors of p21 using a dosing regimen comprising 5 mg/kg to 100 mg/kg for a period of time of 1 day to 6 months, thereby accelerating the healing of the bone fracture in the subject. In one aspect, the one or more inhibitors of p21 comprise UC2288, Butyrolactone I, Sorafenib, LLW10, Daprodustat, Vadadustat, Molidustat, Roxadustat, Desidustat, or combinations thereof. In another aspect, the one or more inhibitors of p21 comprise UC2288 having the structure:

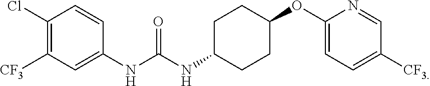 [SMILES: O=C(Nc1ccc(Cl)c(C(F)(F)F)c1)N[C@H]1CC[C@H](Oc2ccc(C(F)(F)F)cn2)CC1]

In another aspect, the one or more inhibitors of p21 suppress p21 gene expression and protein expression in the subject. In another aspect, the one or more inhibitors of p21 suppress p21 gene expression and protein expression in mesenchymal stem cells (MSCs) in the subject. In another aspect, suppression of p21 gene expression and protein expression in MSCs in the subject reduces adipogenic differentiation, increases osteogenic capacity, and increases chondrogenic capacity in the subject. In another aspect, the subject is ≥50 years of age. In another aspect, the subject is ≥75 years of age. In another aspect, the subject is diabetic or has an insulin resistance disorder In another aspect, the one or more inhibitors of p21 are administered to the subject at a situs of the bone fracture, implant, or joint replacement. In another aspect, the one or more inhibitors of p21 are systemically administered to the subject. In another aspect, the dosing regimen comprises a single dose or a plurality of doses of the therapeutically effective amount of the one or more inhibitors of p21.

Another embodiment described herein is a method for regenerating bone or improving bone strength in a subject, the method comprising: administering to the subject a therapeutically effective amount of one or more inhibitors of p21 using a dosing regimen comprising 5 mg/kg to 100 mg/kg for a period of time of 1 day to 6 months, thereby regenerating bone in the subject. In one aspect, the one or more inhibitors of p21 comprise UC2288, Butyrolactone I, Sorafenib, LLW10, Daprodustat, Vadadustat, Molidustat, Roxadustat, Desidustat, or combinations thereof. In another aspect, the one or more inhibitors of p21 comprise UC2288 having the structure:

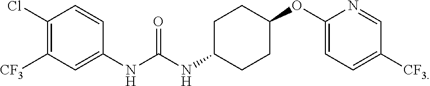 [SMILES: O=C(Nc1ccc(Cl)c(C(F)(F)F)c1)N[C@H]1CC[C@H](Oc2ccc(C(F)(F)F)cn2)CC1]

In another aspect, the one or more inhibitors of p21 suppress p21 gene expression and protein expression in the subject. In another aspect, the one or more inhibitors of p21 suppress p21 gene expression and protein expression in mesenchymal stem cells (MSCs) in the subject. In another aspect, suppression of p21 gene expression and protein expression in MSCs in the subject reduces adipogenic differentiation, increases osteogenic capacity, and increases chondrogenic capacity in the subject. In another aspect, the subject is ≥50 years of age. In another aspect, the subject is ≥75 years of age. In another aspect, the subject is diabetic or has an insulin resistance disorder. In another aspect, the one or more inhibitors of p21 are administered to the subject at a situs for bone regeneration or improving bone strength. In another aspect, the one or more inhibitors of p21 are systemically administered to the subject. In another aspect, the dosing regimen comprises a single dose or a plurality of doses of the therapeutically effective amount of the one or more inhibitors of p21.

Another embodiment described herein is a method for reducing the likelihood of bone loss in an osteoporotic environment or during chemotherapeutic treatment of a subject, the method comprising: administering to the subject a therapeutically effective amount of one or more inhibitors of p21 using a dosing regimen comprising 5 mg/kg to 100 mg/kg for a period of time of 1 day to 6 months, thereby reducing the likelihood of bone loss in the subject. In one aspect, the one or more inhibitors of p21 comprise UC2288, Butyrolactone I, Sorafenib, LLW10, Daprodustat, Vadadustat, Molidustat, Roxadustat, Desidustat, or combinations thereof. In another aspect, the one or more inhibitors of p21 comprise UC2288 having the structure:

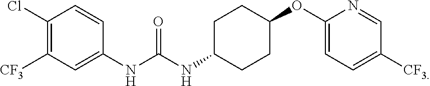 [SMILES: O=C(Nc1ccc(Cl)c(C(F)(F)F)c1)N[C@H]1CC[C@H](Oc2ccc(C(F)(F)F)cn2)CC1]

In another aspect, the one or more inhibitors of p21 suppress p21 gene expression and protein expression in the subject. In another aspect, the one or more inhibitors of p21 suppress p21 gene expression and protein expression in mesenchymal stem cells (MSCs) in the subject. In another aspect, suppression of p21 gene expression and protein expression in MSCs in the subject reduces adipogenic differentiation, increases osteogenic capacity, and increases chondrogenic capacity in the subject. In another aspect, the subject is ≥50 years of age. In another aspect, the subject is ≥75 years of age. In another aspect, the subject is diabetic or has an insulin resistance disorder. In another aspect, the one or more inhibitors of p21 are administered to the subject at a situs of the osteoporotic environment. In another aspect, the one or more inhibitors of p21 are systemically administered to the subject. In another aspect, the dosing regimen comprises a single dose or a plurality of doses of the therapeutically effective amount of the one or more inhibitors of p21. In another aspect, the one or more inhibitors of p21 are systemically administered to the subject with a chemotherapeutic.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed.

Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for accelerating the healing of a bone fracture, implant, or joint replacement in a subject, the method comprising:
   administering to the subject a therapeutically effective amount of one or more inhibitors of p21 using a dosing regimen comprising 5 mg/kg to 100 mg/kg for a period of time of 1 day to 6 months, thereby accelerating the healing of the bone fracture in the subject.
Clause 2. The method of clause 1, wherein the one or more inhibitors of p21 comprise UC2288, Butyrolactone I, Sorafenib, LLW10, Daprodustat, Vadadustat, Molidustat, Roxadustat, Desidustat, or combinations thereof.
Clause 3. The method of clause 1 or 2, wherein the one or more inhibitors of p21 comprise UC2288 having the structure:

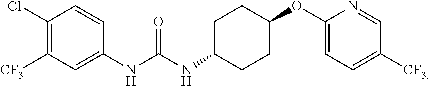 [SMILES: O=C(Nc1ccc(Cl)c(C(F)(F)F)c1)N[C@H]1CC[C@H](Oc2ccc(C(F)(F)F)cn2)CC1]

Clause 4. The method of any one of clauses 1-3, wherein the one or more inhibitors of p21 suppress p21 gene expression and protein expression in the subject.
Clause 5. The method of any one of clauses 1-4, wherein the one or more inhibitors of p21 suppress p21 gene expression and protein expression in mesenchymal stem cells (MSCs) in the subject.
Clause 6. The method of any one of clauses 1-5, wherein suppression of p21 gene expression and protein expression in MSCs in the subject reduces adipogenic differentiation, increases osteogenic capacity, and increases chondrogenic capacity in the subject.
Clause 7. The method of any one of clauses 1-6, wherein the subject is ≥50 years of age.
Clause 8. The method of any one of clauses 1-7, wherein the subject is ≥75 years of age.
Clause 9. The method of any one of clauses 1-8, wherein the subject is diabetic or has an insulin resistance disorder.
Clause 10. The method of any one of clauses 1-9, wherein the one or more inhibitors of p21 are administered to the subject at a situs of the bone fracture, implant, or joint replacement.
Clause 11. The method of any one of clauses 1-10, wherein the one or more inhibitors of p21 are systemically administered to the subject.
Clause 12. The method of any one of clauses 1-11, wherein the dosing regimen comprises a single dose or a plurality of doses of the therapeutically effective amount of the one or more inhibitors of p21.
Clause 13. A method for regenerating bone or improving bone strength in a subject, the method comprising:
   administering to the subject a therapeutically effective amount of one or more inhibitors of p21 using a dosing regimen comprising 5 mg/kg to 100 mg/kg for a period of time of 1 day to 6 months, thereby regenerating bone in the subject.
Clause 14. The method of clause 13, wherein the one or more inhibitors of p21 comprise UC2288, Butyrolactone I, Sorafenib, LLW10, Daprodustat, Vadadustat, Molidustat, Roxadustat, Desidustat, or combinations thereof.
Clause 15. The method of clause 13 or 14, wherein the one or more inhibitors of p21 comprise UC2288 having the structure:

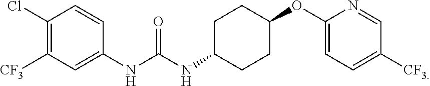 [SMILES: O=C(Nc1ccc(Cl)c(C(F)(F)F)c1)N[C@H]1CC[C@H](Oc2ccc(C(F)(F)F)cn2)CC1]

Clause 16. The method of any one of clauses 13-15, wherein the one or more inhibitors of p21 suppress p21 gene expression and protein expression in the subject.
Clause 17. The method of any one of clauses 13-16, wherein the one or more inhibitors of p21 suppress p21 gene expression and protein expression in mesenchymal stem cells (MSCs) in the subject.
Clause 18. The method of any one of clauses 13-17, wherein suppression of p21 gene expression and protein expression in MSCs in the subject reduces adipogenic differentiation, increases osteogenic capacity, and increases chondrogenic capacity in the subject.
Clause 19. The method of any one of clauses 13-18, wherein the subject is ≥50 years of age.
Clause 20. The method of any one of clauses 13-19, wherein the subject is ≥75 years of age.
Clause 21. The method of any one of clauses 13-20, wherein the subject is diabetic or has an insulin resistance disorder.
Clause 22. The method of any one of clauses 13-21, wherein the one or more inhibitors of p21 are administered to the subject at a situs for bone regeneration or improving bone strength.
Clause 23. The method of any one of clauses 13-22, wherein the one or more inhibitors of p21 are systemically administered to the subject.
Clause 24. The method of any one of clauses 13-23, wherein the dosing regimen comprises a single dose or a plurality of doses of the therapeutically effective amount of the one or more inhibitors of p21.
Clause 25. A method for reducing the likelihood of bone loss in an osteoporotic environment or during chemotherapeutic treatment of a subject, the method comprising:
   administering to the subject a therapeutically effective amount of one or more inhibitors of p21 using a dosing regimen comprising 5 mg/kg to 100 mg/kg for a period of time of 1 day to 6 months, thereby reducing the likelihood of bone loss in the subject.
Clause 26. The method of clause 25, wherein the one or more inhibitors of p21 comprise UC2288, Butyrolactone I, Sorafenib, LLW10, Daprodustat, Vadadustat, Molidustat, Roxadustat, Desidustat, or combinations thereof.
Clause 27. The method of clause 25 or 26, wherein the one or more inhibitors of p21 comprise UC2288 having the structure:

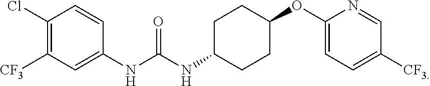 [SMILES: O=C(Nc1ccc(Cl)c(C(F)(F)F)c1)N[C@H]1CC[C@H](Oc2ccc(C(F)(F)F)cn2)CC1]

Clause 28. The method of any one of clauses 25-27, wherein the one or more inhibitors of p21 suppress p21 gene expression and protein expression in the subject.
Clause 29. The method of any one of clauses 25-28, wherein the one or more inhibitors of p21 suppress p21 gene expression and protein expression in mesenchymal stem cells (MSCs) in the subject.
Clause 30. The method of any one of clauses 25-29, wherein suppression of p21 gene expression and protein expression in MSCs in the subject reduces adipogenic differentiation, increases osteogenic capacity, and increases chondrogenic capacity in the subject.
Clause 31. The method of any one of clauses 25-30, wherein the subject is ≥50 years of age.
Clause 32. The method of any one of clauses 25-31, wherein the subject is ≥75 years of age.
Clause 33. The method of any one of clauses 25-32, wherein the subject is diabetic or has an insulin resistance disorder.
Clause 34. The method of any one of clauses 25-33, wherein the one or more inhibitors of p21 are administered to the subject at a situs of the osteoporotic environment.
Clause 35. The method of any one of clauses 25-34, wherein the one or more inhibitors of p21 are systemically administered to the subject.
Clause 36. The method of any one of clauses 25-35, wherein the dosing regimen comprises a single dose or a plurality of doses of the therapeutically effective amount of the one or more inhibitors of p21.
Clause 37. The method of any one of clauses 25-36, wherein the one or more inhibitors of p21 are systemically administered to the subject with a chemotherapeutic.

### EXAMPLES

### Example 1

### Materials and Methods

### Bone Marrow Cell Isolation

All procedures with animals were approved by the IACUC at the University of Wisconsin-Milwaukee. The mice used for this study were C57BL/6 (Charles River, MA). Fifteen 8-week-old male mice were euthanized by CO2 inhalation. After checking for a heartbeat, a cervical dislocation was performed followed by checking for any signs of breathing again for precautionary measures. After confirming that there were no signs of breathing, the tibia and the femur were extracted and placed in phosphate buffered saline (PBS) (Corning Inc., NY). A bone marrow flush with a 23-gauge, 0.5-inch needle (Becton Dickinson, NJ) was performed in a biosafety hood and the pelleted bone marrow cell suspension was plated in a 60×15 mm Primaria cell culture dish (Corning Inc., NY) in MesenCult Basal Medium (Mouse) that contained 50 mL MesenCult 10× Supplement (Mouse) and 0.5 mL MesenPure 1000× (STEMCELL Technologies, Vancouver, Canada). The plates were incubated at 37° C. in a 5% CO2 humidified atmosphere. After 24 hours, half the medium volume was removed, and fresh medium was added. The media in the cultures were renewed every other day. As the cells reached confluency, they were detached by treatment with 0.5% Trypsin-EDTA (Thermo Fisher Scientific, MA) and replated into a 100×20 mm Primaria cell culture dish (Corning Inc.) and eventually into a T-25-cm² Primaria flask (Corning Inc, NY).

### Purification of Mesenchymal Stem Cells (MSCs)

Once the cells were 90-100% confluent in a T-25 flask, they were washed in PBS and trypsinized for 5 minutes in the incubator. DMEM was then added to the cells to neutralize the trypsin and the solution was transferred to a 15 mL falcon tube (VWR, PA). The tubes were centrifuged at 1400 rpm for 7 minutes at 14-16° C. to form a cell pellet. In the meantime, the magnetic buffer was prepared by diluting 1 mL BD IMag Buffer (10×) (BD Biosciences, NJ) in 9 mL UltraPure distilled H₂O (Life Technologies, CA) to form a 1:10 dilution. After the centrifugation process was completed, the supernatant was dumped in a waste container and the cells were resuspended in 1 mL of diluted buffer solution. 5 μL of the BD IMag Biotin Mouse Lineage Depletion Cocktail (BD Biosciences, NJ) was added to the 1 mL of cells and vortexed at a speed of 6 or less. The cells were then placed on ice for 15 minutes. 5 L of BD IMag Streptavidin Particles (BD Biosciences, NJ) was added to cells and mixed gently.

The cells were placed on ice again for 15 minutes. The cell suspension was then transferred to a falcon 5 mL vial round bottom test tube (Corning Inc., NY) and 1 mL of the diluted buffer solution was added. The vial was placed in a magnet for 7 minutes. After 7 minutes, a brown tinge was seen towards the side of the vial closest to the magnet. All the liquid was pipetted out without disturbing the Streptavidin Particles (brown tinge/particles) and transferred to another falcon tube which was then centrifuged. The supernatant was discarded, and the cell pellet was gently broken up with 1 mL of the buffer solution. 2 μL of Ly-6A/E (Sca-1) (Invitrogen, MA) was added, gently mixed, and placed on ice for 15 minutes. 2 μL of the Streptavidin Particles were added to the cells and were mixed gently before being placed back on ice for another 15 minutes. After 15 minutes, the solution was transferred to the 5 mL vial and 1 mL of the buffer solution was added and placed in the magnet for 7 minutes. This time the liquid was discarded, and the brown tinge/particles were saved by resuspending in Dulbecco's Modified Eagle Medium (DMEM) (Thermo Fisher Scientific, MA) with 10% fetal bovine serum (Thermo Fisher Scientific, MA), 1% MEM NEAA (Thermo Fisher Scientific, MA), and 1% Anti-Anti (Thermo Fisher Scientific, MA).

### Cytotoxicity (MTT Assay)

In order to test the cytotoxicity of UC2288 at different concentrations, specifically at 2.5 μM, 5 μM, and 10 μM, an MTT assay was performed. UC2288 (Abcam, UK) was diluted in DMSO, and the aliquots were stored at −20° C. To make a solution at a concentration of 2.5 μM, 5 μM, and 10 μM, 1 μL of the drug solution was added to 8 mL, 4 mL, and 2 mL of DMEM, respectively. A 12 mM stock solution of 3-(4,5-dimethylthiazol-2-yl)-2,5-diphenyl-2H-tetrazolium bromide (MTT) was prepared by diluting the 5 mg of MTT (Thermo Fisher Scientific, MA) in 1 mL PBS. The purified MSCs were seeded in a 96-well plate at a cell density of 5000 cells/well. Each drug concentration was tested in triplicates i.e., there were 3 wells of cells per concentration. An additional 3 wells were used as the positive control group where cells were incubated in DMEM instead of the different drug concentrations. The cells were suspended in 500 μL of the specific drug concentration and incubated at 37° C. for a period of 24-hours and 48-hours. After the respective incubation periods, the medium was replaced with 100 μL PBS and 10 μL of the 12 mM MTT stock solution was added to each well. A negative control group was included by simply adding PBS in 3 new wells without cells with 10 μL of MTT stock solution. The 96-well plates were incubated at 37° C. for four hours after which all but 25 μL of medium was removed from each well. 50 μL of dimethyl sulfoxide (DMSO) (Sigma-Aldrich, MO) was added to the wells and mixed thoroughly with a pipette. The plates were then incubated for 10 minutes, and the samples were read at an absorbance of 540 nm using an Infinite 200 PRO spectrophotometer (Tecan, Männedorf, Switzerland).

### Histology

In order to test whether UC2288 can improve osteogenic capacity of MSCs, the purified MSCs were differentiated into osteoblasts after interaction with UC2288. For osteogenic differentiation experiment, 84,000 cells were added into wells of a 24-well plate (Corning Inc., NY). Each concentration of UC2288, i.e., 2.5 μM, 5 μM, and 10 μM were tested in triplicates along with their controls which had no drugs but simply DMEM media and incubated for a period of 24- and 48-hours. After the designated time periods, the drug solutions were aspirated out of each well. To test for osteogenesis at different concentrations, relevant media were added to cells and renewed every other day for up to 21 days.

The osteogenic media consisted of 1 nM of dexamethasone, 50 μg/mL of L-Ascorbic acid, and 10 mM of β-glycerophosphate (Sigma-Aldrich, MO) in 50 mL DMEM.

After 21 days of culture, cells were stained following the appropriate staining protocol. To prepare the Alizarin Red S staining solution for osteogenic differentiation, 2 g of Alizarin Red S (Sigma-Aldrich, MO) was dissolved in 100 ml distilled water and the pH was adjusted to 4.1-4.3 with HCl or NH₄OH. The cells were washed with PBS and incubated at room temperature in NBF for at least 30 minutes. NBF was aspirated after 30 minutes, and the cells were washed with distilled water. Alizarin Red S staining solution was added to cover the cell monolayer and incubated at room temperature in the dark for 45 minutes. The cells were then washed four times with 1 ml distilled water and kept in PBS. The differentiated osteoblasts feature vast extracellular calcium deposits. Therefore, the calcium deposits are an indication of successful differentiation which stain bright orange-red using Alizarin Red S. These bright orange-red stained cells were observed under the microscope.

### Example 2

### Cytotoxicity

An MTT assay was carried out because higher concentrations of UC2288 drug have previously been reported to cause cytotoxicity in some cell types.

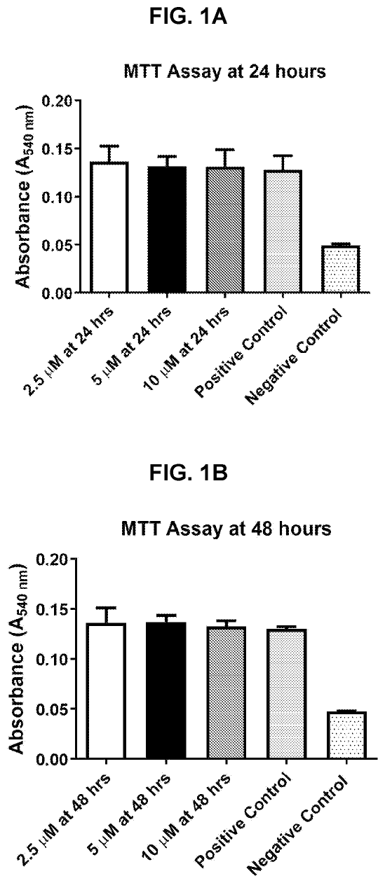

The MTT assay was conducted at concentrations of 2.5 M, 5 μM, and 10 μM UC2288 in MSCs (FIG. 1A-B). The cells were incubated for 24 hours in the specific drug concentrations before reading the plate at an absorbance of 540 nm (FIG. 1A). Here, the positive control were wells that contained cells without any drug concentrations, i.e., plain DMEM media was used. In the negative control group, no cells were added to the wells. The wells contained PBS and MTT. No significant difference was observed for the proliferation of the MSCs with the UC2288 drug and the MSCs with the positive control DMEM media. The different UC2288 drug concentrations also did not have a significant impact on the proliferation of the MSCs.

Similarly, another MTT assay was conducted at treatment concentrations of 2.5 μM, 5 μM, and 10 μM UC2288 for 48 hours (FIG. 1B). The experiment was set up identically to the 24-hour MTT experiment with the only difference being that the incubation period was set up for a longer time, i.e., the cells were incubated in the drug concentrations for 48 hours. No significant difference was observed for the proliferation of the MSCs with the UC2288 drug and the MSCs with the positive control DMEM media. The different UC2288 drug concentrations also did not have a significant impact on the proliferation of the MSCs.

### Example 3

### Histology to Determine Osteogenic Differentiation

This experiment tested the ability of cells that interacted with the UC2288 drug to generate bone-forming osteoblast cells. The effects of UC2288 on MSCs were assessed to determine which concentrations, if any, resulted in the highest number of osteoblasts. After staining each well by following an osteogenic staining protocol, the area of the stains was analyzed using ImageJ and the data were graphed in GraphPad Prism.

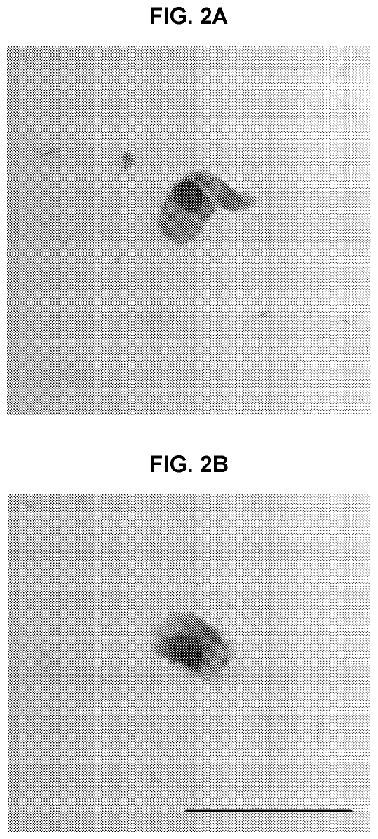

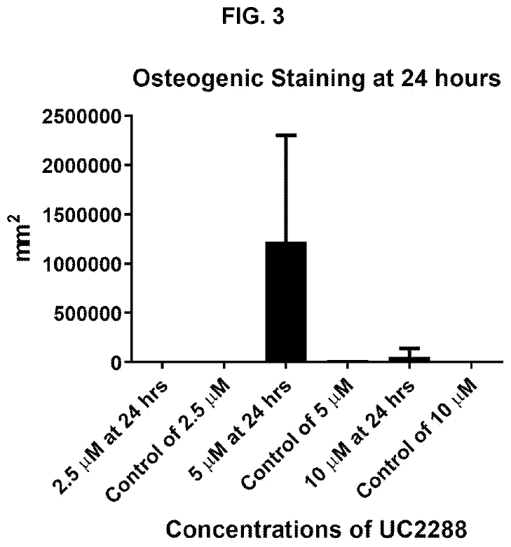

FIG. 2A-B show representative images of the effects of UC2288 treatment by staining for calcium deposits in osteoblasts from MSCs. FIG. 2A shows MSCs treated with 10 μM UC2288 for 24 hr. FIG. 2B shows MSCs treated with the control of DMEM media. Greater staining was observed for MSCs treated with 10 μM UC2288. FIG. 3 shows a bar graph showing the mean and standard deviation of the area of osteogenic stained cells at 2.5 μM, 5 M, and 10 μM of UC2288 treatment for 24 hr, along with the controls. Here, the 5 μM concentration of UC2288 at 24 hr had the most osteogenic stained cells, which supported the hypothesis that UC2288 can improve osteogenic capacity of MSCs.

### Example 4

### MicroCT Imaging of Fracture Healing in Mice Following Post-Fracture Administration of UC2288

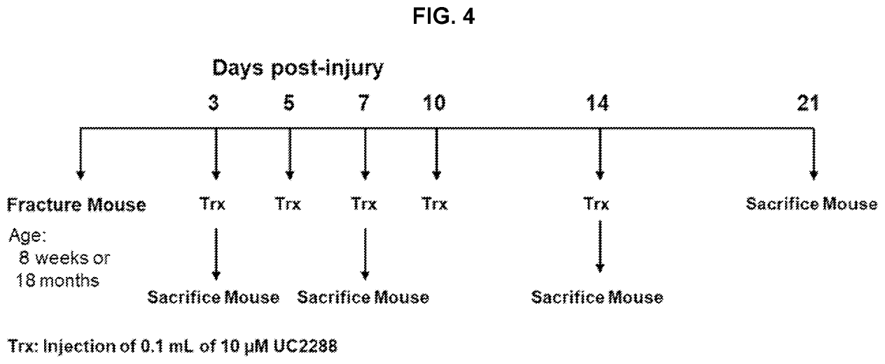

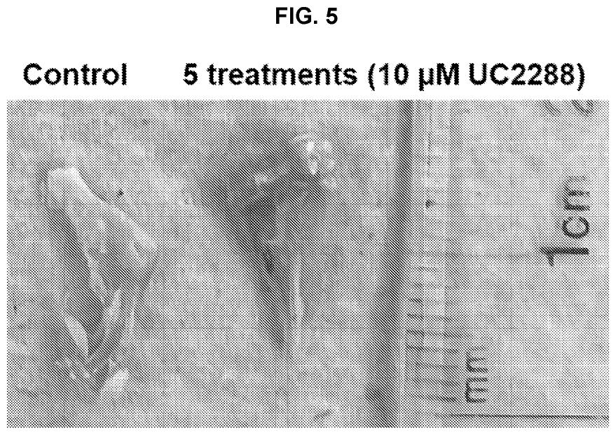

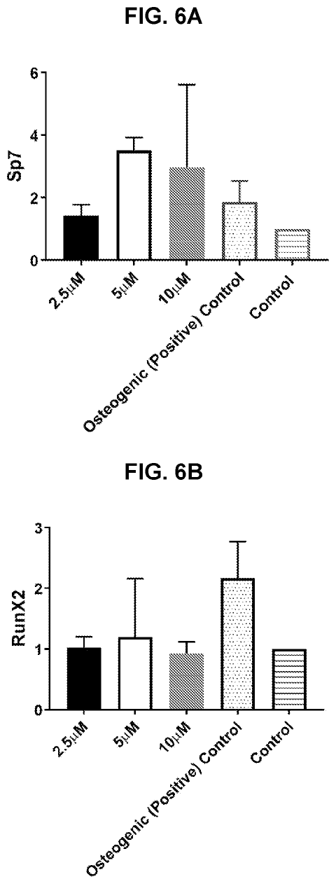

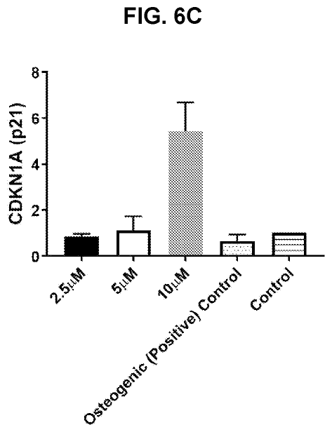

Male wildtype mice (n=10) aged between 8-10 weeks were selected for this study. Future experiments will also include aged mice (e.g., ˜18 months of age). FIG. 4 shows a schematic of the experimental workflow for the study. The mice had their tibiae fractured at the beginning of the study. Five mice were considered controls and received five injections of saline solution at days 3, 5, 7, 10, and 14 post-injury. The other five mice were injected five times with 0.1 mL 10 μM of UC2288 solution at days 3, 5, 7, 10, and 14 post-injury. At 21 days post-injury, mice were sacrificed, and their tibiae were excised. These bones were imaged using a microCT system that can differentiate between bone and soft tissue. FIG. 5 shows representative images of the soft tissue and bone volume differences in fractured tibiae of mice treated with saline control and UC2288. Mice injected with the five 10 μM doses of UC2288 showed more bone formation/healing than the control mice.

Further experiments test whether administering the p21 suppressor UC2288 mediates improved bone healing in an aged wildtype mouse model. The ability of UC2288 to suppress p21 in aged mesenchymal stem cells (MSCs) in vitro followed by characterizing chondrogenic and osteogenic capacity of p21-suppressed aged MSCs compared to young MSCs is analyzed. Drug toxicity is assessed at different concentrations and osteo-chondro differentiation capacity is examined via qPCR and a subcutaneous implant model. Transverse fracture healing is assessed using bone healing via histology at 5-, 14-, and 28-days post-fracture in aged wildtype mice administered with either UC2288 or saline control. The aged wildtype mice treated with UC2288 should demonstrate improved bone healing because p21 is suppressed and these mice are comparable to young mice with respect to their p21 expression.

### Example 5

### Gene Expression Analysis

Human umbilical cord derived mesenchymal stem cells (HuMSCs) were plated in a 24 well plate at a density of 30,000 cells per well. Samples were divided as controls—no drug, no differentiation media; osteogenic control—no drug, DMEM media supplemented with factors to induce osteogenesis including dexamethasone, L-ascorbic acid, and beta-glycerol phosphate; 2.5, 5, and 10 μM wells consisted of DMEM media supplemented respective concentrations of UC2288. Media was renewed in all wells every other day. After 7 days, all samples were processed to extract total RNA. Next, cDNA was produced and qPCR was performed for gene expression analyses. Three genes of interest were analyzed-RunX2, Sp7 (Osterix), and CDKN1A (p21). RunX2 and Sp7 are genes responsible for osteoblast differentiation and take part in osteogenesis. To analyze the data, the ΔΔCₜ method was employed against an 18s endogenous control and DMEM media without differentiation factors or drugs as reference controls. The ΔΔCₜ can be described as:

Relative mRNA expression of sample=2^{-ΔΔCt of sample},

where ΔΔCₜ of sample=(Cₜ of sample−Cₜ of 18s of sample)−(Cₜ of reference control−Cₜ of 18s of reference control).

### Example 6

### Microtomography Imaging

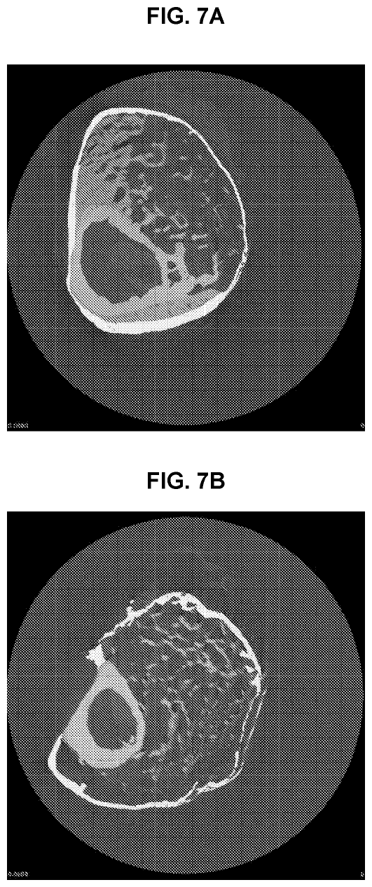

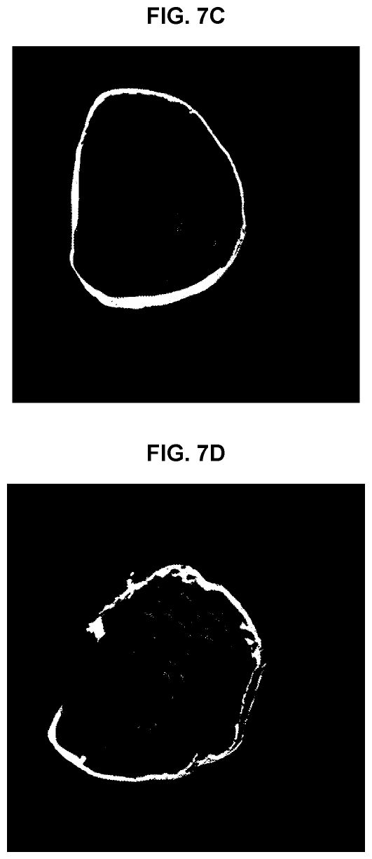

All protocols were approved by IACUC at UWM. C57BI/6 wildtype mice at 8 weeks of age were administered a transverse tibial fracture. The mice were injected with 10 μM of UC2288 solution at a volume of 0.1 mL at days 3, 5, 7, 10, and 14. The mice were sacrificed 4 weeks post-fracture and their tibiae were excised. The bone samples were placed in 10% formalin for two days followed by placement in 70% ethanol until they were ready for microCT imaging. Each sample was placed on a holder and few drops of phosphate buffered saline were placed along with the sample. The sample was scanned using a microCt (Zeiss Xradia Versa X-ray microscope) scanner at a 15 μm thickness per slice. The data was contoured using MIMICS (Materialise NV) followed by data analysis via Python. The blue contour in the sample and control images shows callus formation in the area of fracture. The green contour shows bone being regenerated. See FIG. 7A-B. The black and white images show where bone was automatically thresholded via ImageJ. See FIG. 7D-C. After analysis, it was determined that the injected sample had a callus surface area of 0.114675848 mm² and the control sample had a pixel area in the callus area of 0.094888642 mm². The injected sample had a callus volume of 0.420617666 mm³ and the control sample had a callus volume of 0.277479999 mm³. See Table 2.

TABLE 2
microCT Data for UC2288 Treatment versus Control

| UC2288 Injection | | | | Control | | | |
|---|---|---|---|---|---|---|---|
| Surface Area | Volume | Surface Area | Volume | Surface Area | Volume | Surface Area | Volume |
| Pixels | Pixels | (mm²) | (mm³) | Pixels | Pixels | (mm²) | (mm³) |
| 7611878.877 | 27919486 | 0.114675848 | 0.420617666 | 6298456.621 | 18418387 | 0.094888642 | 0.277479999 |

### Example 7

### Determination of Optimal Drug Concentrations to Reverse Aged MSCs to Young MSCs by Suppressing p21

The drug UC2288 should suppress p21 in aged MSCs, bringing p21 expression close to that of young MSCs. Drug concentrations of 0.1, 1, 2.5, 5, and 10 μM are tested for various time points on MSCs harvested from bone marrow flushes of aged C57BL/6 mice. Following drug treatment, the cells are tested for p21 expression via qPCR and western blot and compared to untreated cells as well as MSCs harvested from young C57BL/6 mice. Post-treatment MSCs are fixed and tested for cytoplasmic versus nuclear localization of p21 via in situ immunofluorescent staining.

### Example 8

### Characterization of the Differentiation Capacity in Drug-Treated Aged MSCs Versus Untreated and Young MSCs

Drug-treated aged MSCs should demonstrate reduced adipogenic and improved osteo-chondro differentiation capacity similar to young MSCs. Drug-treated aged MSCs, aged MSCs, and young MSCs are cultured in osteogenic, chondrogenic, and adipogenic differentiation media followed by qPCR and histology to determine osteo/chondro/adipogenic genes and calcium deposits/proteoglycan or GAG expression/lipid deposits, respectively. A subcutaneous implant model is used to show that the osteogenic capacity of aged MSC can be improved with p21 inhibition.

### Example 9

### Characterization of Fracture Healing in Aged C57BL/6 Mice Following Post-Fracture Administration of UC2288

Administration of UC2288 should enhance bone regeneration in aged mice. Following fracture, UC2288 is administered at the site of injury and bone and cartilage content are quantified temporally and spatially. microCT scanning and histology are used to quantify bone and cartilage content. The methods are similar to those described in Premnath et al., BMC Musculoskeletal Disord. 18: 435 (2017).

### Example 10

### Characterization of Bone Loss/Formation in Ovariectomized Mice

UC2288 should protect against bone loss in an ovariectomized environment. Mice are ovariectomized and treated with varying concentrations of UC2288. Next, bones are analyzed via micro-CT analysis and histology. The experiments are similar to those described in Premnath et al., PLOS One 14(4): e0215018 (2019).

### Example 11

### Characterization of the Effects of UC2288 on Different Bone Healing Modes

UC2288 should improve bone healing in both intramembranous ossification and endochondral ossification where intramembranous ossification is a stabilized form of healing and endochondral ossification is an unstabilized injury. The model used is similar to that described in Thompson et al., J. Orthop. Res. 20 (5): 1091-1098 (2002). Following interaction with UC2288 drug, bones are analyzed via histology and microCT imaging.

### Example 12

Proteomics to Assess Cellular and Molecular Pathways that are Affected by UC2288

It is expected that UC2288 alters several other molecular pathways within MSCs. The extent to which these pathways play a role in bone formation/bone healing is assessed. The MSCs are treated with 0.1, 1, 2.5, 5, or 10 μM of UC2288 for 24, 48, and 72 hours. After incubation with and without drugs, genomic expression is characterized to determine if protein/gene expression is altered with and without interaction with the drug.

### Example 13

### Influence of p21 Overexpression and Inhibition of Bone Healing

A p21 expression-increasing drug is used to assess if p21 overexpression results in less bone healing, as it is expected that there is less bone formation when p21 is overexpressed. The model used is similar to that described in Thompson et al., J. Orthop. Res. 20 (5): 1091-1098 (2002). Following interaction with the p21 expression-increasing drug, bones are analyzed via histology and microCT imaging to assess the level of bone healing/formation.

### Example 14

### Improved Wound Healing Through Inhibition of p21 in Young, Old, and Diabetic Mouse Models

In all three mouse models induce full-thickness excisional wounds are induced in the middle of the dorsum using a biopsy punch. The diameter of the wounds varies between 1-4 cm where 1 cm is considered non-critical, and 4 cm is a critical defect. A p21 inhibiting drug, such as UC2288, is administered at the site of injury and the regeneration of tissue and speed to regeneration of tissue are monitored through digital photographs and histological and immunofluorescence analyses.

### Example 15

Determining Whether p21 Inhibiting Drugs have a Role in Bone Resorption in Osteoclasts

Monocytes are extracted from mice bone marrow. They are plated in petri dishes and treated with different concentrations of p21 inhibitors such as UC2288. Their capacity to differentiate into osteoclasts is measured. Next, a resorption assay is carried out to assess the resorption capacity of osteoclasts that have been treated with UC2288 versus ones that have not been treated.

### Example 16

### p21 Inhibitor's Role in Generating Brittle or Ductile Bones

Mice are treated with varying concentrations of p21 inhibiting drugs after a fracture. Healing bones will be extracted at weeks 2, 4, 6, and 8. The bone is tested using a nanoindentor. Stress, strain values are calculated using the Oliver-Pharr method. See Oliver and Pharr, J. Material Res. 7:1564-1583 (1992). From the stress-strain curves, it is determined whether animals treated with UC2288 regenerate bones that are more brittle or ductile.

### Example 17

### p21 Inhibiting Drugs Versus Other Chemotherapy Drugs

p21 inhibiting drugs are tested with and against and with other commonly used chemotherapy drugs such as paclitaxel and tamoxifen. It has been shown in clinical trials and literature that chemotherapy drugs can cause loss of bone over time. Different doses of p21 inhibiting drugs, such as UC2288, and chemotherapy drugs are tested to ascertain whether the p21 inhibitors protect against bone loss when used alone or in combination with chemotherapy drugs to treat cancer.

What is claimed:

1. A method for accelerating the healing of a bone fracture, implant, or joint replacement in a subject, the method comprising:
administering to the subject a therapeutically effective amount of one or more inhibitors of p21 using a dosing regimen comprising 5 mg/kg to 100 mg/kg for a period of time of 1 day to 6 months, thereby accelerating the healing of the bone fracture in the subject.

2. The method of claim 1, wherein the one or more inhibitors of p21 comprise UC2288, Butyrolactone I, Sorafenib, LLW10, Daprodustat, Vadadustat, Molidustat, Roxadustat, Desidustat, or combinations thereof.

3. The method of claim 2, wherein the one or more inhibitors of p21 comprise UC2288 having the structure:

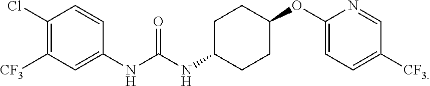 [SMILES: O=C(Nc1ccc(Cl)c(C(F)(F)F)c1)N[C@H]1CC[C@H](Oc2ccc(C(F)(F)F)cn2)CC1]

4. The method of claim 1, wherein the one or more inhibitors of p21 suppress p21 gene expression and protein expression in the subject.

5. The method of claim 4, wherein the one or more inhibitors of p21 suppress p21 gene expression and protein expression in mesenchymal stem cells (MSCs) in the subject.

6. The method of claim 5, wherein suppression of p21 gene expression and protein expression in MSCs in the subject reduces adipogenic differentiation, increases osteogenic capacity, and increases chondrogenic capacity in the subject.

7. The method of claim 1, wherein the subject is ≥50 years of age.

8. The method of claim 7, wherein the subject is ≥75 years of age.

9. The method of claim 1, wherein the subject is diabetic or has an insulin resistance disorder

10. The method of claim 1, wherein the one or more inhibitors of p21 are administered to the subject at a situs of the bone fracture, implant, or joint replacement.

11. The method of claim 1, wherein the one or more inhibitors of p21 are systemically administered to the subject.

12. The method of claim 1, wherein the dosing regimen comprises a single dose or a plurality of doses of the therapeutically effective amount of the one or more inhibitors of p21.

13. A method for regenerating bone or improving bone strength in a subject, the method comprising:
administering to the subject a therapeutically effective amount of one or more inhibitors of p21 using a dosing regimen comprising 5 mg/kg to 100 mg/kg for a period of time of 1 day to 6 months, thereby regenerating bone in the subject.

14. The method of claim 13, wherein the one or more inhibitors of p21 comprise UC2288, Butyrolactone I, Sorafenib, LLW10, Daprodustat, Vadadustat, Molidustat, Roxadustat, Desidustat, or combinations thereof.

15. The method of claim 14, wherein the one or more inhibitors of p21 comprise UC2288 having the structure:

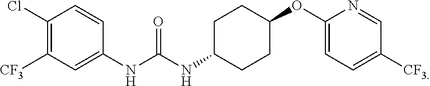 [SMILES: O=C(Nc1ccc(Cl)c(C(F)(F)F)c1)N[C@H]1CC[C@H](Oc2ccc(C(F)(F)F)cn2)CC1]

16. The method of claim 13, wherein the one or more inhibitors of p21 suppress p21 gene expression and protein expression in the subject.

17. The method of claim 16, wherein the one or more inhibitors of p21 suppress p21 gene expression and protein expression in mesenchymal stem cells (MSCs) in the subject.

18. The method of claim 17, wherein suppression of p21 gene expression and protein expression in MSCs in the subject reduces adipogenic differentiation, increases osteogenic capacity, and increases chondrogenic capacity in the subject.

19. The method of claim 13, wherein the subject is ≥50 years of age.

20. The method of claim 19, wherein the subject is ≥75 years of age.

21. The method of claim 13, wherein the subject is diabetic or has an insulin resistance disorder.

22. The method of claim 13, wherein the one or more inhibitors of p21 are administered to the subject at a situs for bone regeneration or improving bone strength.

23. The method of claim 13, wherein the one or more inhibitors of p21 are systemically administered to the subject.

24. The method of claim 13, wherein the dosing regimen comprises a single dose or a plurality of doses of the therapeutically effective amount of the one or more inhibitors of p21.

25. A method for reducing the likelihood of bone loss in an osteoporotic environment or during chemotherapeutic treatment of a subject, the method comprising:
administering to the subject a therapeutically effective amount of one or more inhibitors of p21 using a dosing regimen comprising 5 mg/kg to 100 mg/kg for a period of time of 1 day to 6 months, thereby reducing the likelihood of bone loss in the subject.

26. The method of claim 25, wherein the one or more inhibitors of p21 comprise UC2288, Butyrolactone I, Sorafenib, LLW10, Daprodustat, Vadadustat, Molidustat, Roxadustat, Desidustat, or combinations thereof.

27. The method of claim 26, wherein the one or more inhibitors of p21 comprise UC2288 having the structure:

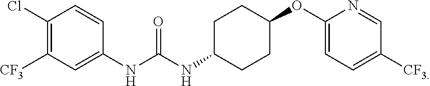 [SMILES: O=C(Nc1ccc(Cl)c(C(F)(F)F)c1)N[C@H]1CC[C@H](Oc2ccc(C(F)(F)F)cn2)CC1]

28. The method of claim 25, wherein the one or more inhibitors of p21 suppress p21 gene expression and protein expression in the subject.

29. The method of claim 28, wherein the one or more inhibitors of p21 suppress p21 gene expression and protein expression in mesenchymal stem cells (MSCs) in the subject.

30. The method of claim 29, wherein suppression of p21 gene expression and protein expression in MSCs in the subject reduces adipogenic differentiation, increases osteogenic capacity, and increases chondrogenic capacity in the subject.

31. The method of claim 25, wherein the subject is ≥50 years of age.

32. The method of claim 31, wherein the subject is ≥75 years of age.

33. The method of claim 25, wherein the subject is diabetic or has an insulin resistance disorder.

34. The method of claim 25, wherein the one or more inhibitors of p21 are administered to the subject at a situs of the osteoporotic environment.

35. The method of claim 25, wherein the one or more inhibitors of p21 are systemically administered to the subject.

36. The method of claim 25, wherein the dosing regimen comprises a single dose or a plurality of doses of the therapeutically effective amount of the one or more inhibitors of p21.

37. The method of claim 25, wherein the one or more inhibitors of p21 are systemically administered to the subject with a chemotherapeutic.
